(19) United States
(12) Patent Application Publication
(10) Pub. No.: US 2025/0195754 A1
(43) Pub. Date: Jun. 19, 2025

(54) REUSABLE CONTROL ASSEMBLY AND PRODUCT-DELIVERY DEVICE COMPRISING SUCH AN ASSEMBLY

(71) Applicant: Nemera La Verpillière, La Verpilliere (FR)

(72) Inventor: Sébastien DELVALAC, Lyon (FR)

(73) Assignee: Nemera La Verpillière, La Verpilliere (FR)

(21) Appl. No.: 19/072,602

(22) Filed: Mar. 6, 2025

Related U.S. Application Data
(63) Continuation of application No. PCT/EP2023/074560, filed on Sep. 7, 2023.

(30) Foreign Application Priority Data
Sep. 8, 2022 (FR) FR2209005

(51) Int. Cl. A61M 5/142; A61M 5/50

(52) U.S. Cl. CPC A61M 5/14244; A61M 5/50; A61M 2005/14268

(57) ABSTRACT

This reusable control assembly for controlling a product-delivery device for administering a product is capable of being reversibly assembled with a disposable part which comprises at least one securing member capable of reversibly securing these together. The control assembly comprises at least one piston and a piston-moving subassembly, the movement of the piston being intended to drive the product out of a cartridge, and a push-button movable between a first position, in which it does not interact with the securing member, and a second position in which, when the reusable control assembly is assembled with the disposable part, the push-button is able to command the securing member to move in order to disconnect the disposable part from the reusable control assembly. The control assembly also comprises a blocking member for blocking the push-button in its first position.

The present invention relates to a reusable control assembly for controlling a product-delivery device and to a product-delivery device for administering a product, notably a drug.

Advantageously, the present invention relates to a device for administering a product by injection, notably to a portable injection device able to be attached to the body of a user, in contact with their skin, and to administer a product to the user parenterally.

In the field of the treatment of certain afflictions, it is known practice to form a drug-administration device that comprises a reusable first part, such as a reusable control assembly, and a disposable second part, mounted on the reusable first part. Such a structure is known, for example, from documents EP-A-3659645 and WO-A-2021/076540 and allows elaborate and potentially costly elements, notably a control subassembly for controlling a drug-administering pump, to be incorporated into the reusable part, while less-expensive elements and a cartridge containing the product that is to be administered are incorporated into the disposable part.

With this type of equipment, the two parts of the device need to be held together securely while the device is being used. To this end, document EP-A-3659645 teaches the use of a reversible locking mechanism which comprises a tab positioned on one wall of one of the parts of the device and intended to catch on a corresponding element belonging to the other part. An unlocking actuator is provided in order to move the tab into a configuration in which it allows the two parts of the device to be separated.

Sliding a cartridge containing a product that is to be administered, into a hollow housing of a main body, is also known, from U.S. Pat. No. 11,426,523. A wedge-shaped cam operated by an actuator allows a sterile seal to be pierced and a needle to be moved, without influencing the secure attachment between the cartridge and the main body.

With this kind of known device there is a risk of untimely disconnection of the two parts that make up the product-delivery device at a time when the product present in the disposable part has not yet been fully administered, or when the device is still in use, notably while it is affixed against the skin of a patient.

The equipment items known from EP-A-3928814, WO-A-2015/091758, US-A-2010/094251, US-A-2020/254185 and WO-A-2017/060899 all incur comparable disadvantages.

It is these disadvantages that the invention more particularly intends to overcome by proposing a novel reusable control assembly for controlling a product-delivery device for administering a product, the operation of which is more secure.

To this end, the invention relates to a reusable control assembly for controlling a product-delivery device for administering a product, the reusable control assembly being capable of being reversibly assembled with a disposable part of the product-delivery device, the disposable part including at least one securing member capable of reversibly securing together the reusable control assembly and the disposable part. According to the invention, the reusable control assembly comprises:
at least one piston and a piston-moving mechanism, the movement of the piston being intended to drive the product out of a cartridge,
a push-button movable between
a first position, and
a second position in which, when the reusable control assembly is assembled with the disposable part, the push-button is able to command the securing member to move in order to disconnect the disposable part from the reusable control assembly.

Furthermore, the reusable control assembly comprises a blocking member for blocking the push-button in its first position.

Thanks to the invention, the push-button blocking member is able to inhibit the passage of the push-button into its second position in which it is able to move the securing member in order to disconnect the disposable part from the reusable control assembly. In other words, the blocking member ensures that the disposable part cannot be separated from the reusable control assembly even if the push-button is inadvertently activated or fails

According to advantageous but non-compulsory aspects of the invention, such a reusable control assembly incorporates one or more of the following features, considered alone or in any technically permissible combinations:
when it is in its first position and when the reusable control assembly is assembled with the disposable part, the push-button is not engaged with the securing member. Under these same circumstances, the push-button does not interact with the securing member;
the blocking member is able to move between a non-blocking position in which it allows the push-button to pass from its first position toward its second position, and a blocking position, in which it blocks the push-button in its first position;
the reusable control assembly comprises elastic return means returning the blocking member to its blocking position;
the elastic return means comprise a spring, preferably a helical spring mounted around a rod that extends in a direction of travel of the blocking member between its blocking and non-blocking positions;
the piston is configured to command the blocking member to move from its blocking position toward its non-blocking position;
the piston is translationally mobile between a rest position, in which it presses against the blocking member that it holds in its non-blocking position, and a plurality of operating positions in which it allows the blocking member to move relative to its non-blocking position, notably from its non-blocking position toward its blocking position;
the piston comprises a piston rod and a thrust element fixed to the piston rod, the thrust element comprising an abutment surface able to come to press against the blocking member that the thrust element holds in its non-blocking position when the piston is in its rest position;
the reusable control assembly comprises a casing housing the piston and the piston-moving subassembly, the casing comprising a guide surface able to collaborate with a complementary guide surface belonging to the thrust element in order to guide the thrust element as the piston moves toward its rest position;
the guide surface and the complementary guide surface are inclined with respect to an axis of movement of the piston by the one same angle of between 20° and 70° with respect to the axis of movement, preferably of between 30° and 60°;
the reusable control assembly comprises a casing housing the piston and the piston-moving subassembly, while the push-button is translationally mobile with respect to the casing between its first and second positions, in a first direction, and the blocking member is translationally mobile with respect to the casing between its blocking and non-blocking positions, in a second direction not parallel to the first direction and preferably orthogonal to the first direction;
the blocking member comprises an enlarged portion and the push-button comprises a housing to house the widened portion when the push-button is in its first position and the blocking member is in its blocking position;
the blocking member comprises at least one rod providing translational guidance with respect to the casing, and the enlarged portion is a collar surrounding the rod.

Another aspect of the invention relates to a product-delivery device for administering a product and comprising a reusable control assembly as mentioned hereinabove and a disposable part capable of being reversibly assembled with the reusable control assembly.

Advantageously, the disposable part includes the securing member, a product-delivery subassembly for administering the product, and a cartridge containing a product that is to be administered and accepting a sliding plug, and in that the piston is able to cause the sliding plug to move inside a body of the cartridge.

Provision may be made for the securing member to be a hook configured to engage with a portion of the reusable control assembly, the hook being able to move under the action of the push-button.

### BRIEF DESCRIPTION OF THE FIGURES

The invention will be better understood and further advantages thereof will become more clearly apparent in the light of the following description of two embodiments of a reusable control assembly and of a product-delivery device that conform to the principle of this invention, this description being given purely by way of example and making reference to the attached drawings in which:

FIG. 1 is a perspective view of a product-delivery device for administering a product according to a first embodiment of the invention, comprising a reusable control assembly likewise in accordance with a first embodiment of the invention, the delivery device being ready for use;

FIG. 2 is a perspective view analogous to FIG. 1 during the course of the assembling of a disposable part on the reusable control assembly of the device of FIG. 1;

FIG. 3 is a perspective view of the reusable control assembly of FIGS. 1 and 2, with one half-shell of a casing omitted;

FIG. 4 is a view, on a larger scale, of detail IV of FIG. 3, viewed from a different angle and with a push-button and a blocking member depicted in exploded view relative to the rest of the reusable control assembly;

FIG. 5 is a theoretical part-section, on the plane V of FIG. 1, of the reusable control assembly and of a disposable part, when the reusable control assembly is in a first configuration, in which a piston of the control assembly extends into a cartridge of the disposable part and an unlocking force E1 for unlocking the disposable part from the reusable control assembly is applied;

FIG. 6 is a part-section, analogous to FIG. 5, of the reusable control assembly and of the disposable part when the reusable control assembly is in a second configuration in which its piston is retracted relative to the disposable part and the same force E1 is applied; and

FIG. 7 is a view analogous to FIG. 5 for a reusable control assembly and a product-delivery device for administering a product according to a second embodiment of the invention.

### DETAILED DESCRIPTION

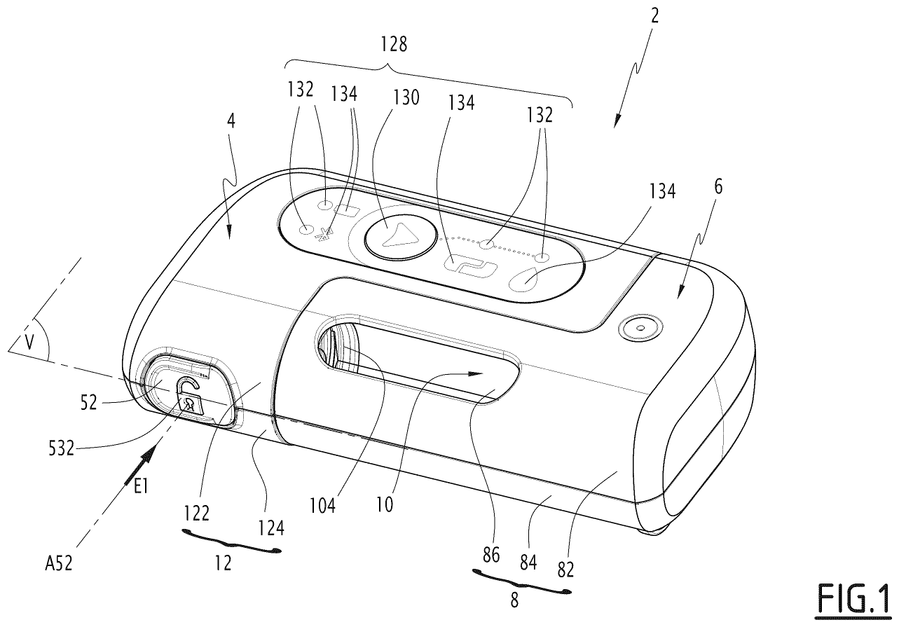

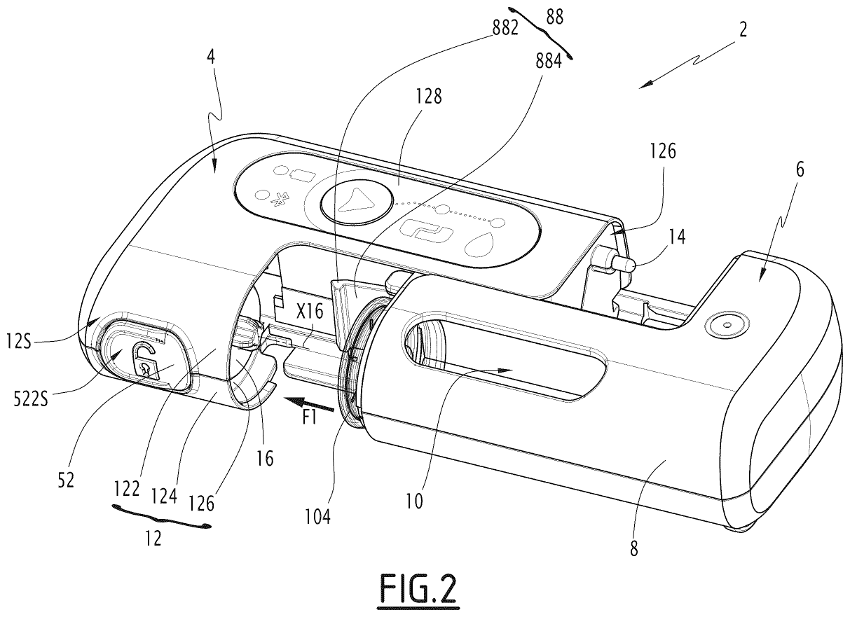

The product-delivery device 2 for administering a product depicted in its entirety in FIGS. 1 and 2 comprises a reusable control assembly 4 and a disposable part 6.

This disposable part 6 comprises a casing 8 formed of two half-shells 82 and 84 which enclose a cartridge 10 of product that is to be administered, this cartridge being partially visible through a window 86 delimited in the half-shell 82 of the casing 8.

The cartridge 10 comprises a body 102, made of glass or of plastic, and a plug 104, also referred to as “piston plunger”, preferably made of elastomer and designed to slide along a longitudinal axis X10 of the cartridge 10 inside the cartridge.

The cartridge 10 is connected, at the opposite end from the plug 104, to a product-delivery subassembly, not depicted, which in particular comprises a needle for injecting the product that is to be administered. The needle is, for example, able to be moved between a retracted position in which it is comprised inside the casing 8 and a deployed position in which it extends out of the casing 8 and is able to be positioned through the skin of a user. This product-delivery subassembly may, for example, be in accordance with the technical teaching of one of the documents WO-A-2021/224388 and WO-A-2022/023391.

The part 6 is intended to be mounted on the control assembly 4 to slide in the direction of the arrow F1 in FIG. 2, to be controlled by the control assembly 4 at the time of administration of the product contained in the cartridge 10 and then be disposed of when the cartridge 10 has been emptied or when the quantity of product that is to be administered that is present in this cartridge reaches a predefined minimum value. Thus, a plurality of disposable parts 6 may be mounted in succession on the control assembly 4 which is therefore reused with each of these different parts 6.

The control assembly 4 comprises a casing 12 formed of two half-shells 122 and 124.

A rod 14 extends from one face 126 of the casing 12 and allows selective activation of the product-delivery subassembly.

The control assembly 4 also comprises a piston 16 configured to drive the sliding plug 104 toward a product-administration end of the cartridge 10, along the axis X10, so as to cause the product to be expelled from the cartridge 10. This piston 16 is visible on a face 126 of the casing 12, visible in FIGS. 2 to 6. Its movement is intended to cause product contained in the cartridge 10 to be administered.

The piston 16 comprises a piston rod 161 and a thrust element 163 fixed to the piston rod.

The front side 16A of the thrust element 163 is defined as being that one of its sides that faces toward the plug 104 when the parts 4 and 6 are assembled to make up the device 2. The rear side 16B of the thrust element 6 is defined as being the side facing in the opposite direction from its front side.

The piston rod 161 is, for example, a flexible rod formed by a coil spring, and the thrust element 163 is intended to be fixed to the spring for example by screwing it into a corresponding end 161A of the spring. The turns of the coil spring form, for example, at this end, a tapping able to collaborate with a corresponding screw thread formed on a stud 164 created on a rear face 165 of the thrust element 163, in an attachment region. In this instance, the thrust element 163 is of one-piece.

The front side 16A of the thrust element 163 is equipped with a tip 162 intended to enter a correspondingly shaped housing 106 formed on a rear side 104B of the plug 104 that faces toward the piston 16 when the product-delivery device 2 is in the configuration for use.

The half-shell 122 is equipped with a control interface 128 which comprises various control buttons 130, and display members 132 consisting of light-emitting diodes or LEDs, each display member being associated with a marking 134 in the form of a logo indicative of a parameter displayed by means of this member. The control interface 128 notably enables control of the product-delivery subassembly of the disposable part 6 via the rod 14, and of a motor 42, via an electronic control board 44.

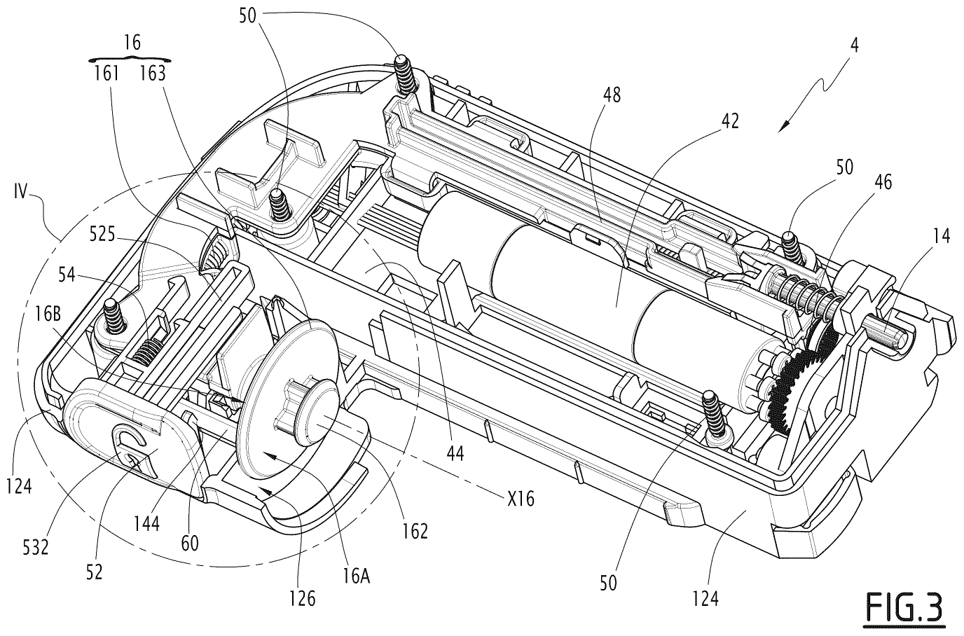

As visible in FIG. 3 in which the half-shell 122 has not been depicted, the casing of the reusable control assembly 4 contains the electric motor 42, the electronic control board 44 and at least one gearset 46 driven by the motor 42 and a mechanism for converting the rotary movement output by the gearset 46 into a translational movement. The movement conversion mechanism is configured to convert the rotary movement output by the gearset 46 into a translational movement of the piston 16, and notably of the thrust element 163, along an axis X16, which is coincident with the axis X10 when the product-administration device 2 is in the assembled configuration. Advantageously, the conversion mechanism is also configured to convert the rotary movement output by the gearset 46 into a translational movement of the rod 14 in order to control the delivery subassembly of the disposable part 6.

The reusable control assembly 4 also comprises a battery, not depicted.

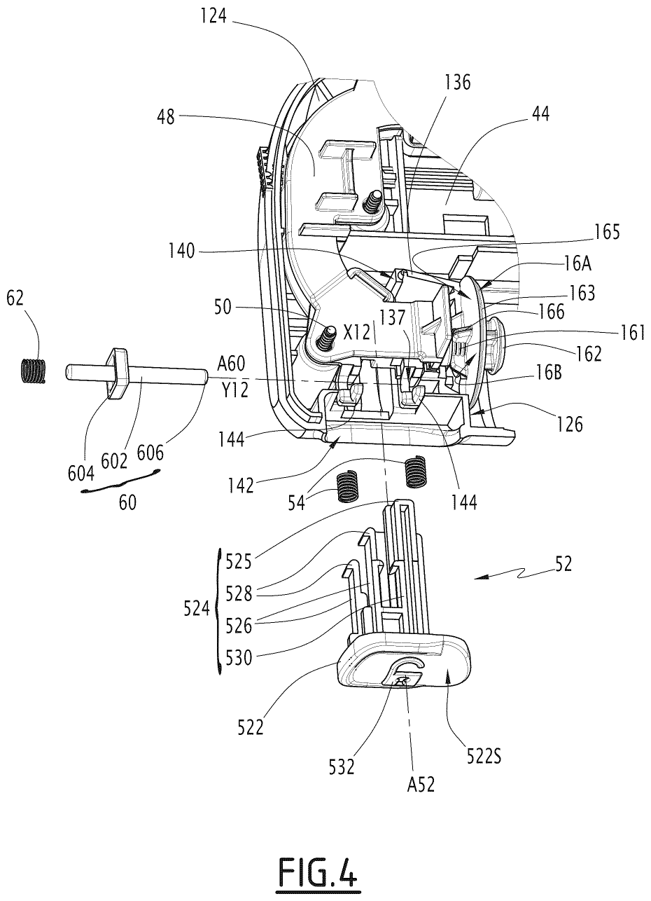

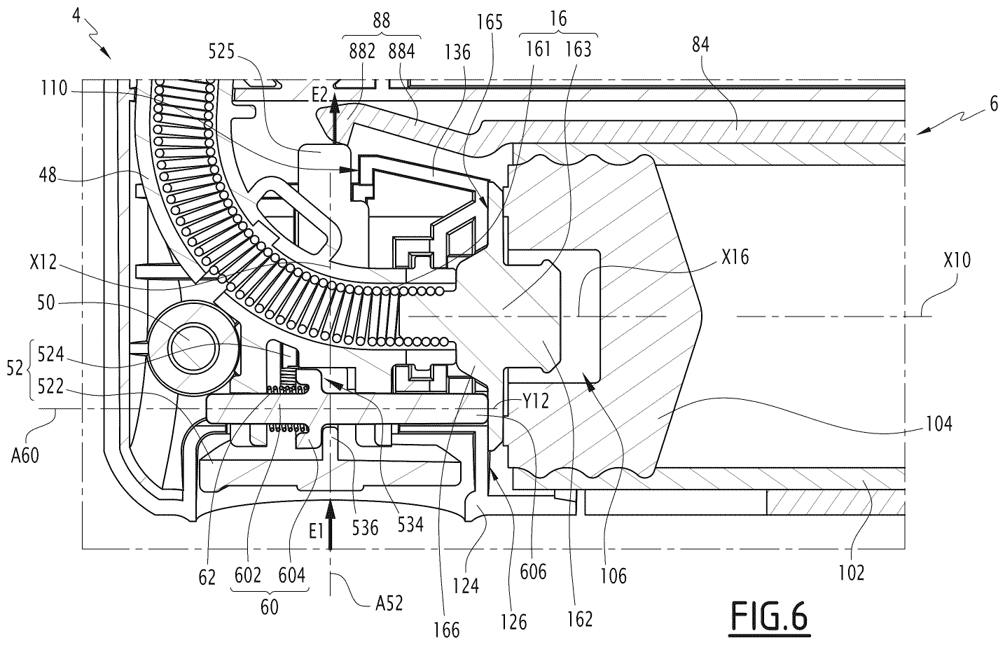

The piston 16 is able to move between a rest position and a plurality of operating positions. In the rest position, depicted particularly in FIGS. 4 and 6, the piston presses against the half-shell 124 of the casing 12 and is advantageously disengaged from the body 102 of the cartridge 10. In an operating position, the piston 16 drives the plug 104 toward the product-administration end of the cartridge 10 and is advantageously engaged in the body 102. There are a plurality of operating positions of the piston 16 distributed along the axis X16.

In the embodiment of FIGS. 1 to 6, the rear side 16B of the thrust element 163 is flat, except in its central part where it bears a relief 166 for centering it with respect to the face 126 of the casing 12. In other words, the rear face 165 of the thrust element 163 comprises a flat annular portion surrounding the relief 166. The stud 164 extends from the relief 166 in the direction away from the front side 16A. The relief 166 is capable of collaborating with a corresponding guide zone belonging to the casing 12 so as to guide the thrust element 163 in its movements from or toward its rest position.

The control board 44 is configured to activate the motor 42 on the basis of orders received by the interface 128 and to return the piston 16 to its rest position when the product contained in the cartridge 10 has been used up in its entirety, or a predetermined quantity has been used up.

The movement conversion mechanism comprises a threaded rod able to be turned by the gearset 46 and a member for controlling the piston 16, capable of translational movement along the threaded rod as the threaded rod turns, in the manner of a screw-nut connection. The control member is fixed to one end of the piston rod 161 which is the opposite end from the end 161A to which the thrust element 163 is fixed, and is able to command the piston rod to move, and thus, during this movement, the thrust element to move. Advantageously, the mechanism comprises a channel 48, configured in the overall shape of a J, able to accept the piston rod 161.

The elements 42 to 48 and the associated battery constitute a piston-moving subassembly for moving the piston 16, notably for moving the thrust element along the axis X16 and for moving the piston rod 161 in the channel 48, in both directions.

The elements that make up the reusable control assembly 4 are relatively elaborate and costly, hence the benefit of reusing them with several disposable parts 6 in succession.

Screws 50 are used to hold the half-shells 122 and 124 securely together. In the embodiment depicted, the screws 50 pass through corresponding housings provided in the half-shell 124 and screw into corresponding tappings in the half-shell 122. Other means of assembling the half-shells with one another are conceivable, notably elastic clip-fastening assembly means.

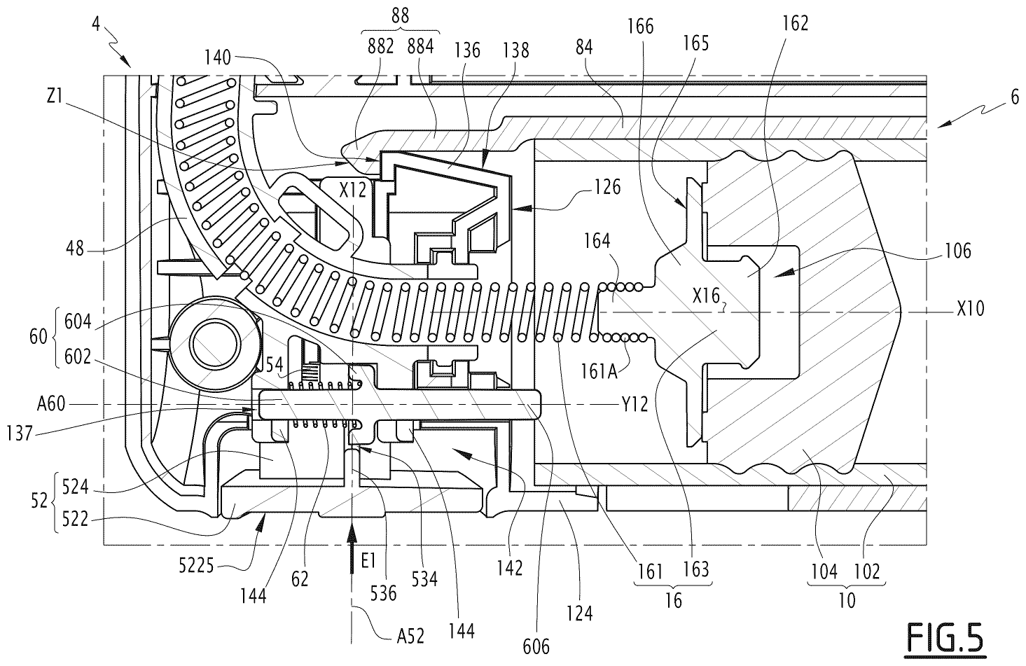

As is more particularly apparent from FIGS. 5 and 6, the casing 8 of the disposable part 6 is equipped with a hook 88 which constitutes a securing member for holding the parts 4 and 6 together when the product-delivery device 2 is in the assembled configuration. The hook 88 is of one piece with the half-shell 84 and comprises a head 882 and a shank 884 connecting the head 882 to the rest of the half-shell 84.

The half-shell 124 also comprises a rigid portion 136 defining a ramp 138 for the sliding of the head 882 during a movement of bringing the parts 4 and 6 closer together in the direction of the arrow F1 in FIG. 2. The rigid portion 136 also defines a catching surface 140, perpendicular to the axis X16, and against which the head 882 comes into abutment when the parts 4 and 6 of the product-delivery device 2 are in the assembled configuration as depicted in FIG. 5.

A push-button 52 is mounted on the casing 12 and comprises a head 522, the exterior shape of which is preferably convex, housed in a housing 142 of corresponding shape, this housing being defined by the two half-shells 122 and 124. In the absence of action by a user on the push-button 52, the exterior surface 522S of the head 522, which is to say that surface of this head that is visible from outside the casing 12, lies flush with an exterior surface 12S of the casing 12.

A longitudinal axis A52 of the push-button 52 is denoted A52.

The push-button 52 also comprises a rod 524 which extends along the longitudinal axis A52 and is made up of a number of prongs of which two prongs 526 are each equipped with a barbed tip 528 and one prong 530 defines the opposite end 525 of the rod 524 from the head 522.

The barbed tips 528 serve to hold the push-button 52 on the casing 12 when the reusable control assembly 4 is in the assembled configuration.

When the reusable control assembly 4 is in the assembled configuration, the axis A52 is superposed with an axis X12 defined by the casing 12 and which is an axis of sliding of the push-button 52 in the casing 12.

By default, the push-button 52 is pushed back by two springs 54 into the position of FIGS. 1, 2 and 5, in which its rod 524 does not interact with the hook 88 and in which the surfaces 522S and 12S are flush with one another.

The external surface 522S of the head 522 bears a marking 532 depicting a padlock to serve as a reminder that the push-button 52 allows the disposable part 6 to be unlocked from the reusable control assembly 4. Specifically, when the head 522 is depressed into the housing 142 by applying to its surface 522S a force E1 directed toward the inside of the casing 122, the push-button 52 exerts, by means of the end 525 of the rod 524, a force E2 that is oriented, and of sufficient strength, to push back the head 882 of the hook 88 so as to disengage it from the catching surface 140.

The movement of the head 882 of the hook 88 under the action of the push-button 52 is achieved as a result of flexural elastic deformation of the shank 884 of the hook 82.

Thus, the push-button 52 has translational mobility, along coincident axes A52 and X12, between a first position depicted in FIG. 5 and a second position depicted in FIG. 6. The first position is different than the second position. In its first position of FIG. 5 and when the parts 4 and 6 are assembled, the push-button 52 is not in contact with the hook 88, which means that it is disengaged from this hook. In other words, in this position, the push-button does not interact with the hook 88. Furthermore, in its first position, the push-button defines, with the catching surface 140, a receiving region Z1 for receiving the hook 88. In its second position, the push-button is situated in the hook-receiving region Z1. Thus, when the parts 4 and 6 are assembled and the push-button is then moved toward its second position, the end 525 of the rod 524 of the push-button pushes the head 882 of the hook 88 back against the effect of an elastic force exerted by the shank 884 to disconnect the disposable part 6 from the reusable control assembly 4.

In other words, the push-button 52 allows the parts 4 and 6 of the product-delivery device 2 to be separated starting from the assembled configuration depicted in FIGS. 1 and 5. The securing-together of the parts 4 and 6 of the product-delivery device 2, that is obtained by means of the hook 88, is thus reversible.

Such disconnection must not be able to be achieved under non-specific conditions. In particular, such disconnection must not be able to be achieved when the piston 16 is engaged in the body 102 of the cartridge 10 because, in that configuration, the piston, or even the mechanism 48, could be damaged during or following separation of the parts 4 and 6. The piston 16 would then be exposed to the outside of the casing 12 and it would not be possible to fit a new disposable part 6, the cartridge 10 of which would be full, on the control assembly 4. Furthermore, only some of the product would have been administered, and in the context of the therapeutic treatment, this is not desirable. In practice, such disconnection is intended to be achieved only after the product contained in the cartridge 10 has been used up in the intended quantity, and after the mechanism 48 driven by the motor 42 has returned the piston 16 to its rest position.

According to the invention, the reusable control assembly 4 comprises a blocking member 60 which allows the push-button 52 to be immobilized in terms of translational movement along the axes A52 and X12, even if a force E1 is exerted on the external face of the push-button 52. This blocking member prevents untimely or accidental disconnection of the parts 4 and 6 of the product-delivery device 2. Thus, the blocking member 60 blocks the push-button 52 in its first position when necessary.

A longitudinal axis of the blocking member 60 is denoted A60.

Advantageously, the blocking member consists of a shuttle 60.

The shuttle 60 is a one-piece part which comprises a rod 602 and a collar 604 that constitutes a portion that is enlarged with respect to the rod 602 and that surrounds this rod. As is more particularly evident from FIG. 4, the collar 604 may have a polygonal, particularly square, cross section. An end of the rod 602 that faces toward the piston 16 is denoted 606. The end 606 may be qualified as the front end of the rod 602.

The shuttle 60 is guided by bearings 144 formed by the half-shell 124 and that define a longitudinal axis Y12 of the housing 137 that houses the shuttle 60. The housing 137 is defined by the casing 12.

Advantageously, the axes X12 and Y12 are perpendicular.

As a variant, the axes X12 and Y12 are non-parallel, without being perpendicular.

In the configuration in which the shuttle 60 is mounted on the half-shell 124, the axes A60 and Y12 are coincident and the shuttle 60 is capable of translational movement along these coincident axes, guided, along the axes Y12 and A60, by the rod 602 collaborating with the bearings 144.

The shuttle 60 is urged by an elastic member 62, in a direction parallel to the axes A60 and Y12, toward a configuration in which the collar 604 is received in a housing 534 defined by the push-button 52.

Advantageously, the elastic force exerted by the elastic member 62 is directed toward the piston 16.

Advantageously, the elastic member 62 is formed by a helical spring made of metal mounted around the rod 602, thereby ensuring uniform application of the elastic return force returning the shuttle 60 toward its position depicted in FIG. 5. This then minimizes the risks of the shuttle 60 becoming wedged.

As a preference, the spring 62 is housed in the housing 534.

As a variant, the elastic member may be a spring of non-helical shape and/or made from a non-metallic material, or else may be an elastic tab of one-piece with the body 12.

The collar 604 and the housing 534 have more or less complementing shapes, and their geometry is defined so that when the collar 604 is received in the housing 534 it blocks translational movement of the push-button 52 in a direction of being depressed into the casing 12, starting from the configuration depicted in FIGS. 1 and 6. The housing 534 is created in the form of a slot passing through a prong 526 that forms part of the rod 524.

Advantageously, the collar is received in the housing 534 with only a small amount of clearance.

According to an invention variant that has not been depicted, the collar may be received in the housing with enough clearance to allow the push-button 52 to be depressed into the casing 12 over a short travel without that causing the end 525 of the rod 524 to push against the head 882.

When its collar 604 is engaged in the housing 534, the shuttle 60 is in a position in which it blocks the push-button 52 in its first position, in which the end 525 of the rod 524 is disengaged from the hook 88. That means that, in its blocking position, the shuttle prevents the parts 4 and 6 of the product-delivery device 2 from being disconnected.

The spring 62 by default returns the shuttle 60 to its blocking position.

When the piston 16 is in the rest position, with the piston rod completely retracted in the channel 48, namely when the piston projects practically not at all with respect to the face 126 of the casing 12, it may be assumed that the intended quantity of product initially contained in the cartridge 10 has been expelled from the cartridge or that this expulsion has not yet been initiated and that, consequently, disconnecting the parts 4 and 6 of the product-delivery device 2 presents no disadvantage.

In this configuration which is depicted in FIG. 6, the rear face 165 of the thrust element 163 comes to press against the end 606 of the rod 602, and this has the effect of pushing the shuttle 60 back against the elastic force exerted by the spring 62 and of driving the collar 604 out of the housing 534.

The rear face 165, particularly the flat annular portion thereof, thus constitutes an abutment surface capable of coming to bear against the shuttle 60 in a configuration in which the thrust element 163 is holding the shuttle in a position of non-blocking of the push-button 52, in which position it is held by the piston 16, as long as the latter remains in its rest position.

In this configuration, the shuttle 60 does not oppose a movement of depressing the push-button 52 into the casing 12, under the effect of the force E1. When such a force is exerted by a user, the push-button is depressed into the body 12 to the extent that the end 525 of the rod 524 is able, thanks to the force E2, to push back the hook 88, which is then disengaged from the catching surface 140. This releases the disposable part 6 from the reusable control assembly 4.

Thus, the piston 16 is configured to command the movement of the shuttle 60 along the coincident axes A60 and Y12. Specifically, if the piston 16 is disengaged from the rod 602, it allows the spring 62 to push the shuttle 60 back toward the configuration depicted in FIG. 5, and the blocking member, which is to say the shuttle, is in the position of blocking the push-button. By contrast, if the piston 60 presses against the end 606 of the rod 602, it pushes the shuttle 60 back out of the housing 534, as depicted in FIG. 6, and the blocking member, which is to say the shuttle, is in the non-blocking position, compatible with movement of the push-button 52 against the action of the force exerted by the springs 54.

In practice, as soon as it moves significantly away from the face 126 of the casing 12, the piston 16, and notably the thrust element 163, allow the shuttle 60 to pass from the non-blocking position of FIG. 6 to the blocking position of FIG. 5.

The position of the shuttle 60 in the configuration of FIG. 5 is a blocking position in which the shuttle 60 blocks the push-button 52 in its first position. The position of the shuttle 60 in the configuration of FIG. 6 is a non-blocking position in which this shuttle allows the push-button 52 to pass from its first position, disengaged from the hook 88, toward its second position in which it pushes the head 882 back with respect to the rigid portion 136.

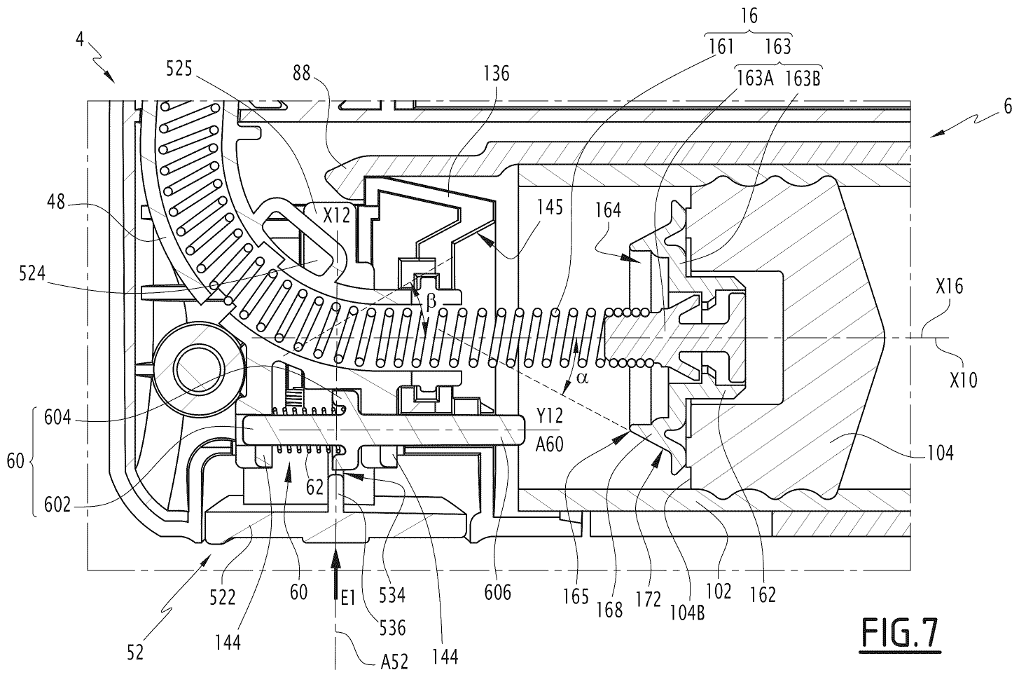

In the second embodiment of the invention, depicted in FIG. 7, the elements that are analogous to those of the first embodiment bear the same references. If a reference is mentioned in the following part of the description, without featuring in FIG. 7, it refers to the same element as the one bearing that same reference in the first embodiment. Likewise, if a reference is featured in FIG. 7, without being mentioned in the description, it refers to the same element as the one bearing that same reference in the first embodiment.

The piston in this second embodiment likewise comprises a rod 161 and a thrust element 163. Here, the thrust element 163 comprises two parts 163A and 163B assembled with one another.

The second embodiment differs from the first embodiment in that, on its rear side 16B, the thrust element 163 is provided with a collar 168 of which the rear edge, on the opposite side from the tip 162, is denoted 165, and the external peripheral surface is denoted 172.

The surface 172 is frustoconical and converges toward the rear of the piston 16, with a vertex half-angle α, this angle being formed between the surface 172 and the axis X16 along which the piston 16 moves.

On the other hand, the rigid portion 136 of the casing 12 defines an internal surface 145 which is frustoconical, centered on the axis X10, and the vertex half-angle of which is denoted β.

The angles α and β are advantageously equal. In other words, the surfaces 145 and 172 are inclined at the same angle with respect to the axis X16.

Thus, the surfaces 145 and 172 constitute guide surfaces that collaborate to guide the thrust element 163, and therefore the piston 16, when it is moved from or toward its rest position, in a comparable way to that depicted in FIG. 6 in respect of the first embodiment.

When the piston 16 is in the rest position, the edge 165 of the collar 168 comes to bear against the end 606 of the rod 602, thereby pushing the collar 604 back out of the housing 534, in an operation comparable to that explained hereinabove in respect of the first embodiment. The rear face 165 also constitutes an abutment surface capable of coming to bear against the shuttle 60 in a configuration in which the thrust element 163 is holding the shuttle 60 in a non-blocking position, as long as the piston 16 remains in its rest position.

In practice, the angles α and β are advantageously of between 20° and 70°, preferably of between 30° and 60°.

As a variant, the surfaces 145 and 172 may be inclined with respect to the axes X10 and X16 without being frustoconical.

Whatever the embodiment, when the piston 16 is in its rest position, it pushes the collar 604 back out of the housing 534, and therefore pushes the shuttle 60 toward a position of non-blocking of the push-button, in which position the force E1 exerted on the head 522 of the push-button 52 enables the hook 88 to be maneuvered. Conversely, as soon as the piston 16 reaches one of its operating positions, the force exerted by the spring 62 pushes the shuttle 60 back from its non-blocking position into a position of blocking of the push-button, in which position the collar 604 is engaged in the housing 534.

Whatever the embodiment, the blocking member 60 may be other than a shuttle. It may have a shape other than that depicted in the figures, and may be not of one piece.

The embodiments and variants envisioned hereinabove may be combined to generate new embodiments of the invention, within the scope of the attached claims.

Whatever the embodiment or variant considered, the product-delivery device according to the invention may notably be used to administer a drug in liquid form to a patient in order to treat a wide variety of conditions, such as, for example, autoimmune diseases or chronic conditions such as diabetes or the associated disorders, cancers, hormone insufficiencies, macular degeneration, inflammation, atherosclerosis, rheumatoid arthritis, coronary syndromes, thromboembolisms, etc.

Thus, the term “drug” refers to any liquid formulation that can be administered continuously by means, for example, of a hollow needle or of a cannula. The formulation may be a solution, a gel or else a fine suspension containing one or more active principles. These active principles may be notably peptides (for example, insulin or GLP-1, their derivatives or analogues, amylin or its derivatives or analogues, gastric inhibitory peptides or their analogues), proteins, glycoproteins or hormones (for example hormones derived from the pituitary gland or the hypothalamus, such as gonadotropin or gonadotropin releasing hormone, desmopressin, gonadorelin, triptorelin, terlipressin, leuprorelin, buserelin, goserelin, nafarelin, lutropin, menotropin, follicle-stimulating hormone and its analogues, follitropin, parathyroid hormone, teriparatide, abaloparatide or other parathyroid hormone analogues, calcitonin, growth hormone, somatropin, etc.), active principles derived from hormones or from nucleotides (for example DNAs, RNAs, oligonucleotides), enzymes, polysaccharides (for example glycosaminoglycans, hyaluronic acids, heparins, low-molecular-weight heparins and their derivatives, or sulfate-or polysulfate-forms of these polysaccharides), vaccines, antibodies and their analogues (for example denosumab, panitumumab, nutritional substances. The formulations may contain these active principles in the form of any pharmaceutically acceptable salt or solvate together with any necessary and appropriate excipient.

1. A reusable control assembly for controlling a product-delivery device for administering a product, the reusable control assembly being capable of being reversibly assembled with a disposable part of the product-delivery device, the disposable part including at least one securing member capable of reversibly securing together the reusable control assembly and the disposable part, wherein the reusable control assembly comprises:
at least one piston and a piston-moving subassembly, the movement of the piston being intended to drive the product out of a cartridge,
a push-button movable between
a first position, and
a second position in which, when the reusable control assembly is assembled with the disposable part, the push-button is able to command the securing member to move in order to disconnect the disposable part from the reusable control assembly,
and in that the reusable control assembly comprises a blocking member for blocking the push-button in its first position.

2. The reusable control assembly as claimed in claim 1, wherein the blocking member is able to move between a non-blocking position in which it allows the push-button to pass from its first position toward its second position, and a blocking position, in which it blocks the push-button in its first position.

3. The reusable control assembly as claimed in claim 2, wherein it comprises an elastic return device returning the blocking member to its blocking position.

4. The reusable control assembly as claimed in claim 3, wherein the elastic return device comprises a spring, preferably a helical spring mounted around a rod that extends in a direction of travel of the blocking member between its blocking and non-blocking positions.

5. The reusable control member as claimed in claim 2, wherein the piston is configured to command the blocking member to move from its blocking position toward its non-blocking position.

6. The reusable control assembly as claimed in claim 2, wherein the piston is translationally mobile between a rest position, in which it presses against the blocking member that it holds in its non-blocking position, and a plurality of operating positions in which it allows the blocking member to move relative to its non-blocking position, notably from its non-blocking position toward its blocking position.

7. The reusable control assembly as claimed in claim 6, wherein the piston comprises a piston rod and a thrust element fixed to the piston rod, the thrust element comprising an abutment surface able to come to press against the blocking member that the thrust element holds in its non-blocking position when the piston is in its rest position.

8. The reusable control assembly as claimed in claim 7, wherein the reusable control assembly comprises a casing housing the piston and the piston-moving subassembly, the casing comprising a guide surface able to collaborate with a complementary guide surface belonging to the thrust element in order to guide the thrust element as the piston moves toward its rest position.

9. The reusable control assembly as claimed in claim 8, wherein the guide surface and the complementary guide surface are inclined with respect to an axis of movement of the piston by the one same angle (α, β) of between 20° and 70° with respect to the axis of movement, preferably of between 30° and 60°.

10. The reusable control assembly as claimed in claim 2, wherein the reusable control assembly comprises a casing housing the piston and the piston-moving subassembly, in that the push-button is translationally mobile with respect to the casing between its first and second positions, in a first direction, and in that the blocking member is translationally mobile with respect to the casing between its blocking and non-blocking positions, in a second direction not parallel to the first direction and preferably orthogonal to the first direction.

11. The reusable control assembly as claimed in claim 2, wherein the blocking member comprises an enlarged portion and in that the push-button comprises a housing to house the widened portion when the push-button is in its first position and the blocking member is in its blocking position.

12. The reusable control assembly as claimed in claim 10, wherein the blocking member comprises at least one rod providing translational guidance with respect to the casing, and in that the enlarged portion is a collar surrounding the rod.

13. A product-delivery device for administering a product, comprising:
a reusable control assembly and
a disposable part capable of being reversibly assembled with the reusable control assembly,
wherein the reusable control assembly is as claimed in claim 12.

14. The product-delivery device for administering a product, as claimed in claim 13, wherein the disposable part includes the securing member, a product-delivery subassembly and a cartridge containing a product that is to be administered and accepting a sliding plug, and in that the piston is able to cause the sliding plug to move inside the body of the cartridge.

15. The product-delivery device for administering a product, as claimed in claim 13, wherein the securing member is a hook configured to engage with a portion of the reusable control assembly, the hook being able to move under the action of the push-button.
